(19) United States
(12) Patent Application Publication
(10) Pub. No.: US 2009/0197968 A1
(43) Pub. Date: Aug. 6, 2009

(54) Extended-release capsules comprising venlafaxine hydrochloride

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 11/658,854

(22) PCT Filed: Aug. 11, 2005
(86) PCT No.: PCT/CA05/01233

§ 371 Date: Jan. 29, 2007

(30) Foreign Application Priority Data
Aug. 12, 2004 (CA) 2476101

(51) Int. Cl. A61K 31/137; A61P 25/24

(52) U.S. Cl. 514/649

(57) ABSTRACT

A capsule comprising venlafaxine hydrochloride wherein part of the drug content is in the form of delayed-release coated spheroids and a second part of the drug content is in a prompt-release form.

### BACKGROUND

Venlafaxine is a drug used for treatment of depression. Venlafaxine and its acid addition salts are disclosed in U.S. Pat. No. 4,535,186. Venlafaxine hydrochloride is sold in the United States and elsewhere under the tradename Effexor™ as tablets, in strengths of 25, 37.5, 50, 75 and 100 mg. The tablets are administered to adults in doses ranging from 75 to 350 mg/day, in divided doses two or three times a day. Upon ingestion of venlafaxine hydrochloride tablets, rapid dissolution results in a rapid increase in blood plasma levels of the active compound, followed by a decrease over several hours, as the active compound is eliminated or metabolized, until subtherapeutic levels are approached about eight to twelve hours following administration, thus requiring further dosing. With the plural daily dosing regimen, the most common side effect is nausea. Vomiting also occurs in some patients.

Venlafaxine hydrochloride is now also sold in the United States and elsewhere, under the tradename Effexor XR™, as extended-release capsules in strengths of 37.5, 75 and 150 mg. These capsules provide gradual release of venlafaxine hydrochloride over a 24-hour period after ingestion, thus enabling a dosing schedule of once daily, while at the same time providing a lower incidence of nausea and vomiting. ™-Trademark.

Effexor XR™ capsules are made in accordance with the disclosure of U.S. Pat. No. 6,274,171. Each capsule contains a multitude of small granules or beads, referred to as “spheroids”. Each spheroid is comprised of a core, and a coating applied to the core. The core is comprised of venlafaxine hydrochloride, microcrystalline cellulose, and hydroxypropylmethylcellulose. The cores are coated with a mixture of ethylcellulose and hydroxypropylmethylcellulose. The ethylcellulose makes the film water-insoluble, while the hydroxypropylmethylcellulose makes the film water-permeable. The result is slow release by permeation through the film, with the release rate dependent on the ratio of hydroxypropylmethylcellulose to ethylcellulose and the thickness of the coat.

The cores are made by a process of mixing the venlafaxine hydrochloride, microcrystalline cellulose, and hydroxypropylmethylcellulose with water to produce a wet plastic mass, which is then extruded, spheronized and dried.

The film coating is then applied by dissolving the ethylcellulose and hydroxypropylmethylcellulose in solvent, and spraying the solution onto the cores in a fluid bed drying system.

According to the disclosure of U.S. Pat. No. 6,274,171, the acceptability of the coating level is determined by the dissolution rate of the coated spheroids using USP Apparatus 1 at 100 rpm in water at 37° C.

Appropriate dissolution specifications are said to be as follows:

| | Average % Venlafaxine |
|---|---|
| Time (hours) | HCl Released |
| 2 | <30 |
| 4 | 30-55 |
| 8 | 55-80 |
| 12 | 65-90 |
| 24 | >80 |

It is further disclosed that capsules made in accordance with the disclosure and meeting these dissolution specifications will result in a peak blood level of venlafaxine at from about four to about eight hours after ingestion.

While capsules according to U.S. Pat. No. 6,274,171 provide a satisfactory extended release product, coating all of the spheroids to reduce the dissolution to below 30% at two hours is costly.

In light of the foregoing, an objective of the present invention is to provide a formulation of extended-release capsules comprising venlafaxine hydrochloride, which does not require coating all of the spheroids to the extent necessary to reduce dissolution of all of the spheroids to below 30% in two hours.

### DESCRIPTION OF THE INVENTION

The present invention is an extended-release formulation of venlafaxine hydrochloride in the form a capsule characterized as follows:
1. From 40% to 70% of the venlafaxine hydrochloride is in the form of coated spheroids, referred to as delayed-release spheroids, which exhibit average dissolution of less than 30% at 2 hours;
2. From 30% to 60% of the venlafaxine hydrochloride is in another form, referred to as a prompt-release form, which exhibits average dissolution of more than 60% at 2 hours. This prompt-release form may be in any of a number of physical forms including, for example, uncoated spheroids, coated spheroids, tablets, or powder; and
3. As a result of containing venlafaxine hydrochloride in both forms, the average dissolution of the capsules, exceeds 30% but is less than 60% at 2 hours.

For purposes of this specification, the dissolution testing is done in USP Apparatus 1 at 100 rpm in 900 mL of phosphate buffer of pH6.8 at 37° C.

The reason for maintaining dissolution of the capsule below 60% at two hours is to reduce the side effects of nausea and vomiting, just as is achieved by compositions of U.S. Pat. No. 6,274,171. The average dissolution of the mixture at two hours will preferably be between 35 and 55%, and will most preferably be about 45%.

As aforesaid, the delayed-release spheroids will be coated spheroids, which will be comprised of core spheroids, to which a coating is applied to delay release. The core spheroids will comprise venlafaxine hydrochloride along with one or more excipients (inactive ingredients). For example, the core spheroids may be made as in U.S. Pat. No. 6,274,171 by mixing venlafaxine hydrochloride with microcrystalline cellulose, hydroxypropylmethylcellulose and water to form a wet plastic mass, extruding, spheronizing, and drying.

A preferred method of making the core spheroids is to use, as an excipient, a water insoluble polymer, such as, for example, ethylcellulose. This enables the core spheroids themselves to exhibit somewhat extended dissolution, so as to reduce the amount of coating required on the core spheroids.

Such core spheroids can be made, for example, by preparing a solution of ethylcellulose in an organic solvent, such as methanol or methylene chloride, mixing the solution into the venlafaxine hydrochloride, drying the wet mass, milling the dried material into granules (i.e. spheroids), and selecting granules of the desired size by sieving.

The coating that is applied to the core spheroids will be a film-coating comprising a water-insoluble polymer, such as, for example, ethylcellulose.

As aforesaid, in addition to containing delayed-release spheroids, the capsules will contain additional venlafaxine hydrochloride in a prompt-release form, which may be in any of a number of physical forms, including, for example, uncoated spheroids, coated spheroids, tablets, or powder.

For example, the core spheroids that are used to make delayed-release coated spheroids may be used, uncoated, as the prompt-release form.

Alternatively, the prompt-release form may consist of the same core spheroids, which, instead of being uncoated, may be coated, but with a lesser amount of coating than the delayed-release spheroids, so as to only slightly delay release.

The capsules of the present invention not only have a lower cost of production than capsules according to U.S. Pat. No. 6,274,171, but also enable greater flexibility of absorption profile, as a result of having the drug present in two forms instead of only one form. The capsules of U.S. Pat. No. 6,274,171 provide a peak venlafaxine blood level at from about 4 to about 8 hours after ingestion. The present invention enables capsules for which the peak venlafaxine blood level is reached in less than 4 hours, but for which the peak level is still no higher than, or not significantly higher than, that obtained with capsules according to U.S. Pat. No. 6,274,171.

The invention will be better understood from the following illustrative examples.

### Example 1

### Core Spheroids

Core spheroids were made as follows:

A quantity of venlafaxine hydrochloride was granulated by adding an equal quantity of ethylcellulose dissolved in methylene chloride, mixing and evaporating the methylene chloride. The resultant dried mass comprised 50% venlafaxine hydrochloride and 50% ethylcellulose. This dried mass was then milled through a #10 screen (10 wires per inch). The milled material was then sifted on a #20 screen. The granules that remained on the #20 screen, having a size from about 850 to about 2000 microns, were then retained for use as core spheroids. As aforesaid, such core spheroids may be used directly, in uncoated form, as the prompt-release form; or they may be coated with a film coat comprising a water insoluble polymer to form delayed-release spheroids.

The average dissolution of these core spheroids was found to exceed 90% at 2 hours when tested in USP Apparatus 1 at 100 rpm in 900 mL of phosphate buffer of pH6.8 at 37° C.

### Example 2

### Delayed-Release Spheroids

600 grams of core spheroids of example 1 were spray-coated with the following coating solution in a fluid bed coating system:

| | |
|---|---|
| Ethylcellulose | 200.0 g |
| Dibulyl Sebeate | 30.0 g |
| Methanol | 1800.0 g |
| | 2030.0 g |
| Total Dry | 230.0 g |

The content of venlafaxine hydrochloride in these delayed-release coated spheroids was 50%×600/830=36.1%. The average dissolution of these delayed-release spheroids was found to be about 15% at 2 hours, when tested in USP Apparatus 1 at 100 rpm in 900 mL of phosphate buffer of pH6.8 at 37° C.

### Example 3

Size 0 two-piece hard gelatin capsules were filled with spheroids as follows:

| | | Venlafaxine |
|---|---|---|
| | Quantity Per | Hydrochloride |
| Capsule | Capsule | Content Per |
| Core spheroids of example 1 | 120.0 mg | 60.0 mg |
| Delayed-release spheroids | 249.0 mg | 90.0 mg |
| of example 2 | 369.0 mg | 150.0 mg |

The average dissolution of these capsules is about 40% to 45% at 2 hours when tested in USP Apparatus 1 at 100 rpm in 900 mL of phosphate buffer of pH6.8 at 37° C.

1. A capsule comprising venlafaxine hydrochloride wherein:
i) from 40% to 70% of the venlafaxine hydrochloride is in coated spheroids, which exhibit average dissolution of less than 30% at 2 hours;
ii) from 30% to 60% of the venlafaxine hydrochloride is in a second form which exhibits average dissolution of more than 60% at 2 hours; and
iii) the average dissolution of the capsule exceeds 30% but is less than 60% at 2 hours.

2. A capsule of claim 1, for which the average dissolution is between 35 and 55% at 2 hours.

3. A capsule of claim 1 wherein the coated spheroids comprise core spheroids, which are coated with a film coating comprising a water-insoluble polymer.

4. A capsule of claim 3 wherein the second form is the uncoated core spheroids.

5. A capsule of claim 1, which exhibits an average time to peak venlafaxine blood level of less than 4 hours after ingestion.
